(19) United States
(12) Patent Application Publication
(10) Pub. No.: US 2002/0193569 A1
(43) Pub. Date: Dec. 19, 2002

(54) Bispecific fusion protein and method of use for enhancing effector cell killing of target cells

(75) Inventor: Nabil Hanna, Rancho Santa Fe, CA (US)

Correspondence Address: PILLSBURY WINTHROP, LLP, P.O. BOX 10500, MCLEAN, VA, 22102, US

(73) Assignee: IDEC Pharmaceuticals Corporation, San Diego, CA

(21) Appl. No.: 09/986,174

(22) Filed: Nov. 7, 2001

(30) Foreign Application Priority Data
Jun. 4, 2001 PCT/US01/40835

(51) Int. Cl. C07K 16/46; A61K 38/21; A61K 39/395

(52) U.S. Cl. 530/351; 530/391.1; 424/85.4; 424/178.1

(57) ABSTRACT

The present invention provides a nucleic acid encoding IFN-α fusion proteins. The specification also describes the proteins encoded by said nucleic acids, methods of making IFN-α fusion proteins and methods of utilizing the compounds for the treatment of cancer. One specific IFN-α fusion protein comprising IFN-α or a portion thereof capable of enhancing the killing ability of an effector cell and an anti-CD20 antibody, such as RITUXAN®, which can target, for example, B cell lymphomas (e.g., non-Hodgkin's lymphoma).

### FIELD OF THE INVENTION

This invention relates to a fusion protein comprising all or a biologically active portion of an interferon alpha (IFN-α) linked to an immunoglobulin protein or polypeptide fragment thereof, which recognizes a cell surface protein expressed by a malignant cell. The fusion protein, when bound to a malignant cell, or a target cell, would bind via IFN-α to the IFN-α receptor expressed on an effector cell (e.g., natural killer (NK) cells, polymorphonuclear (PMNs) cells and macrophages/monocytes). The binding of IFN-α fusion protein to its receptor on effector cells enhances and potentiates extracellular (e.g., antibody-dependent cell-mediated cytotoxicity or ADCC-type), intracellular (phagocytic) and/or direct killing of the bound target cell.

### BACKGROUND OF THE INVENTION

The cytokines, interleukin-2 (IL-2) and IFN-α, are potent activators of natural killer (NK) cells and other anti-tumor effector cells, but results obtained in clinical trials with these cytokines have proved disappointing in many forms of cancer. The ineffectiveness of IL-2 and IFN-α may be because intratumoral monocytes/macrophages (MO) inhibit the cytokine-induced activation of cytotoxic effector lymphocytes, such as NK cells, at the site of tumor growth (Hellstrand et al., Acta Oncol. 37: 347-53 (1998)). Nevertheless, IFN-α can regulate NK cells and lymphokine-activated killer (LAK) cells and can act in synergy with IL-2 to augment NK activity (Chikkala et al., Cancer Res. 50: 1176-82 (1990)). For example, systemically administered IFN-α has exhibited a limited anti-tumor effect in lymphomas, leukemia, Kaposi's sarcoma, renal cell carcinoma, melanoma, multiple melanoma, glioma and ovarian cancer. See Sell et al., Immunology, Immunopathology & Immunity 951-2 (1996). However, systemic treatment with either or both IFN-α or IL-2 can cause less important severe life-threatening toxicity, which limits their administration at higher doses leading to reduced efficacy and discourages their therapeutic use (Meseri-Delwail et al., Biotechnol. Ther. 5: 47-57 (1994); and Sosman et al., Semin. Oncol. 17: 22-30, 38-41 (1990)). Use of IFN-α is further complicated as multiple species of IFN-α exist that differ dramatically in activity, which is explained in part by different binding affinities (Webb et al., Cell Immunol. 124: 158-7 (1989); and U.S. Pat. No. 4,780,530 (1988)). Furthermore, systemic activation of NK cells and monocytes is less effective than activation at the tumor site since activated cells may not home to the tumor. This is particularly applicable to solid tumors including carcinomas.

Antibody dependent cellular cytotoxicity (ADCC) is viewed as an important mechanism by which monoclonal antibodies (mAb) can exert an antitumor effect in vivo. ADCC can be enhanced by the cytokines IL-2 and IFN-α (Vuist et al., Cancer Immunol. Immunother. 36: 163-70 (1993)).

Monoclonal antibodies have been designed to target specific cells or antigens. Monoclonal antibodies therefore, have been designed to act as vectors or delivery vehicles for targeting foreign antigens to cells. See for general description EP Patent No. 553,244 (1993).

Anti-CD19 Antibodies. Antibodies have been raised which recognize CD19, a signal transduction molecule restricted to the B-cell lineage. Examples of monoclonal anti-CD19 antibodies include anti-B4 (Goulet et al. Blood 90: 2364-75 (1997)), B43 and B43 single-chain Fv (FVS191; Li et al., Cancer Immunol. Immunother. 47:121-130 (1998)). Myers et al. (Leuk. Lymphoma 29: 329-38 (1998) reported conjugating the murine monoclonal B43 to the tyrosine kinase inhibitor (see also U.S. Pat. No. 5,872,459), genistein, to produce an immunoconjugate against CD19 antigen positive hematologic malignancies. Treon et al., Semin, Oncol 26/5 Supp: 97-106 (1999) reported conjugation of B4 to a blocked ricin, which had no significant activity in patients with multiple myeloma.

Rituximab and other Anti-CD20 Antibodies. The FDA approved anti-CD20 antibody, Rituximab (IDEC C2B8; RITUXANÔ; ATCC No. HB 11388) has also been used to treat humans. Ibritumomab, is the murine counterpart to Rituximab (Wiseman et al., Clin. Cancer Res. 5: 3281s-6s (1999)). Other reported anti-CD20 antibodies include the anti-human CD20 mAb 1F5 (Shan et al., J. Immunol 162: 6589-95 (1999)), the single chain Fv anti-CD20 mouse mAb 1H4 (Haisma et al., Blood 92: 184-90 (1998)) and anti-B1 antibody (Liu et al., J. Clin. Oncol. 16: 3270-8 (1998)). In the instance of 1H4, a fusion protein was created reportedly fusing 1H4 with the human β-glucuronidase for activation of the prodrug N-[4-doxorubicin-N-carbonyl(-oxymethyl)phenyl] O-β-glucuronyl carbamate to doxorubicin at the tumor cite (Haisma et al. 1998).

Anti-CD22 Antibodies. CD22 is a cell surface antigen expressed on normal human B cells and some neoplastic B cells. Several monoclonal anti-CD22 antibodies have been created, including HD6, RFB4, UV22-2, Tol5, 4KB128, a humanized anti-CD22 antibody (hLL2), and a bispecific F(ab′)₂ antibody linked to saporin (Li et al. Cell. Immunol. 111: 85-99 (1989); Mason et al., Blood 69: 836-40 (1987); Behr et al., Clin. Cancer Res. 5: 3304s-14s (1999); and Bonardi et al., Cancer Res. 53: 3015-21 (1993)). Immunotoxins comprising anti-CD22 linked to the ricin A chain have also been prepared, which reportedly possess antitumor effects in mice (Sausville et al. Blood 85: 3457-65 (1995); and Ghetie et al., Cancer Res. 51: 5876-80 (1991)).

Anti-CD33 Antibodies. CD33 is a glycoprotein expressed on early myeloid progenitor and myeloid leukemic (e.g., acute myelogenous leukemia, AML) cells, but not on stem cells. An IgG₁ monoclonal antibody was prepared in mice (M195) and also in a humanized form (HuM195), that reportedly has antibody-dependent cellular cytotoxicity (Kossman et al., Clin. Cancer Res. 5: 2748-55 (1999)). However, Caron et al., Clin. Cancer Res. 1: 63-70 (1995) reported that HuM195 had only modest ADCC capability against HL60 cells. An anti-CD33 immunoconjugate (CMA-676) consisting of a humanized anti-CD33 antibody linked to the antitumor antibiotic calicheamicin reportedly demonstrated selective ablation of malignant hematopoiesis in some AML patients (Sievers et al., Blood 93: 3678-84 (1999). Pagliaro et al., Clin. Cancer Res. 4: 1971-6 (1998) described a HuM195-gelonin immunoconjugate, comprising an anti-CD33 mAb conjugated to the single-chain plant toxin gelonin.

Anti-CD38 Antibodies. CD38 is an antigen expressed during early stages of differentiation in normal and leukemic myeloid cells, including myeloma cells. Ellis et al., J. Immunol. 155: 925-37 (1995) reported a high affinity mAb (AT13/5) against CD38 which efficiently directed antibody-dependent cellular cytotoxicity (ADCC) against CD38+cell lines, but which activated complement poorly and did not down-regulate CD38 expression. Flavell et al., Hematol. Oncol. 13: 185-200 (1995) described an anti-CD38/anti-saporin (OKT10-Sap) immunotoxin, which purportedly delivers the ribosome inactivating protein (rip) to the leukemic cells (HSB-2 cells). The anti-CD38 antibody, HB7, has been chemically conjugated to a modified ricin molecule and can supposedly kill antigen-bearing tumor cells (Goldmacher et al., 84: 3017-25 (1994)).

Anti-EGF-R Antibodies. Epidermal growth factor-receptor (EGF-R) binds to EGF, a mitogenic peptide. Anti-EGF-R antibodies and methods of preparing them can be performed as described in U.S. Pat. Nos. 5,844,093; 5,558,864. European Patent No. 706,799A purportedly describes an immunoconjugate comprising an anti-EGF-R mAb fused to a C-X-C chemokine, especially IL-8. U.S. Pat. No. 5,824,782 describes an immunoconjugate comprising an anti-EGFR antibody fused to IL-8, which lacks at least the first amino acid of IL-8.

Anti-HM1.24 Antibodies. HM1.24 is a type II membrane glycoprotein is overexpressed in multiple myeloma (MM) and Waldenstrom's macroglobulinemia (Ohtomo et al., Biochem. Biophys. Res. Commun. 258: 583-91 (1999); and Goto et al., Blood 84: 1922-30 (1994)). A mouse monoclonal anti-HM1.24 IgG₂ₐ/κ antibody has been demonstrated to bind to HM1.24 on MM cells and reportedly induces ADCC (Ono et al., Mol. Immuno. 36: 387-95 (1999)). A humanized anti-HM1.24 IgG₁/κ antibody also was shown to induce ADCC against human myeloma KPMM2 and ARH77 cells (Ono et al., Mol. Immuno. 36: 387-95 (1999)).

Anti-Her-2 Antibodies. The ergB 2 gene, more commonly known as (Her-2/neu), is an oncogene encoding a transmembrane receptor. Several antibodies have been developed against Her-2/neu, including trastuzumab (e.g., HERCEPTIN®; Fornier et al., Oncology (Huntingt) 13: 647-58 (1999)), TAB-250 (Rosenblum et al., Clin. Cancer Res. 5: 865-74 (1999)), BACH-250 (Id.), TA1 (Maier et al., Cancer Res. 51: 5361-9 (1991)), and the mAbs described in U.S. Pat. Nos. 5,772,997; 5,770,195 (mAb 4D5; ATCC CRL 10463); and U.S. Pat. No. 5,677,171. Portions of anti-Her-2 antibodies have also been conjugated to toxins, such as a single chain antibody domain specific for erbB-2 coupled to part of a Pseudomonas exotoxin (Skrepnik et al., Clin. Cancer Res. 2: 1851-7 (1996)). U.S. Pat. No. 5,855,866 reported a method of treating the vasculature of solid tumors using an anti-p185^{Her-2} antibody linked to a cytokine.

Anti-MUC-1 Antibodies. MUC-1 is a carcinoma associated mucin. The anti-MUC-1 monoclonal antibody, Mc5, was reportedly administered to mice carrying transplanted breast tumors and purportedly suppressed tumor growth (Peterson et al., Cancer Res. 57: 1103-8 (1997)). Mc5 was chemically linked in a non-cleavable fashion to a natural IFN-α (nIFN-α) and reportedly inhibited growth of injected tumors in mice (Ozzello et al., Breast Cancer Res. Treat. 25: 265-76 (1993)). An IgG₄ anti-MUC-1 mAb, hCTMO1, has been suggested as a suitable carrier for cytotoxic agents in ovarian carcinomas (Van Hof et al., Cancer Res. 56: 5179-85).

Anti-phosphatidyl-serine antigen Antibodies. Phosphatidyl-serine is a phospholipid. Antibodies have been reported which bind to phosphatidyl-serine and not other phospholipids (e.g., Yron et al., Clin. Exp. Immol. 97: 187-92) (1994)). However, anti-phospholipid antibodies appear more typically associated with anti-phospholipid syndrome and its diagnosis, than for use in the treatment or diagnosis of cancer.

Anti-TAG-72 Antibodies. TAG-72 is a tumor-associated antigen (TAG). One dimeric single-chain Fv antibody construct of monoclonal CC49 recognizes the TAG-72 epitope (Pavlinkova et al., Clin. Cancer Res. 5: 2613-9 (1999)). Additional anti-TAG-72 antibodies include B72.3 (Divgi et al., Nucl. Med. Biol. 21: 9-15 (1994)) and those disclosed in U.S. Pat. No. 5,976,531. Administration of recombinant IFN-α reportedly increased the amount of TAG-72 expressed on tumors (Macey et al., Clin. Cancer Res. 3: 1547-55 (1997)). The CC49 antibody also has reportedly been chemically conjugated to doxorubicin (Johnson et al., Anticancer Res. 15: 1387-93 (1995)) and streptavidin (Ngai et al., Nucl. Med. Biol. 22: 77-86 (1995)). TAG-72 has also been chemically conjugated to human interleukin-2 (IL-2) (LeBerthon et al., Cancer Res. 51: 2694-8 (1991). U.S. Pat. No. 5,976,531 claimed a human anti-Tag-72 antibody conjugated to an interferon but no evidence was presented indicating its efficiency in mammals.

Interferon-α Immunoconjugates. Monoclonal antibodies raised against tumor cell lines have been covalently coupled with purified human lymphoblastoid IFN-α. Administration of this coupled form of IFN-α reportedly augmented killing of the tumor cells and other tumor targets by peripheral blood NK cells (Flannery et al., Eur. J. Cancer Clin. Oncol. 20: 791-8 (1984)).

Techniques also have been developed to produce chimeric antibodies which combine regions of immunoglobulin molecules from different sources (Morrison et al., Proc. Natl. Acad. Sci. USA 81: 6851-5 (1984)). Using this technology, chimeric molecules comprising immunoglobulin and non-immunoglobulin regions have been created. Expression vectors for fusion proteins comprising DNA sequences of an antibody and DNA sequences encoding a cell surface antigen have been proposed, wherein some of the suitable antigens include CD molecules (e.g., CD19, CD20, CD22, CD33, CD38 and CD40). See, e.g., U.S. Pat. No. 5,637,481 (1997) and EP Patent No. 610,046 (1994). Fusion proteins comprising the cytokine IL-15 and anti-CD20 (International PCT Application 98/16254), and fusion proteins comprising other lymphokines (e.g., IL-2 and IL-3) and an immunoglobulin fragment capable of binding to a tumor antigen have been described (U.S. Pat. No. 5,645,835 (1997) and U.S. Pat. No. 5,314,995 (1994); Lode et al., Blood 91: 1706-1715 (1998); Hassan et al., Leuk. Lymphoma 20: 1-15 (1995)). Chang et al., U.S. Pat. No. 5,723,125 describes a hybrid molecule comprising an interferon, preferably INF-α-2a or INF-α-2b, which are joined at the carboxy terminus via a peptide to the amino terminus of a first gamma immunoglobulin Fc fragment as a means of increasing the blood half-life of the cytokine. Chimeric immunoglobulin proteins comprising a cytokine (e.g., IL-2, tumor necrosis factor α, etc.) and the heavy chain of an antibody for treating viral infections and cancer are described in International PCT Application 92/08495, as well as a fusion protein of IFN-γ/M-CSF. A fusion protein of RM4/IFN-tau has demonstrated antitumor activity in mice (Qi et al., Hum. Antibodies Hybridomas 7: 21-6 (1996); and Xiang et al., Hum. Antibodies Hybridomas 7: 2-10 (1996)).

Others have proposed the creation of fusion proteins comprising non-antibody immunomodulatory molecules, wherein IFN-α is operatively fused to a heterologous membrane attachment domain (U.S. Pat. No. 5,891,432 (1999)). INF-α-2b was reported to be conjugated with ME31.3 and anti-carcinoembryonic antigen (CEA) and purportedly may improve diagnostic and therapeutic potential of monoclonal antibodies making them worthy of further study (Thakur et al., J. Immunother. 20: 194-201 (1997)).

Therefore, not withstanding what has been previously reported in the literature, there exists a need for improved compositions and methods of using such compositions for the treatment of cancer. Improved immunoconjugates with enhanced ADCC and phagocytotic activities, lower toxicity, and increased blook serum half-life would provide additional treatment therapeutics, which are still needed in cancer therapy.

### SUMMARY OF THE INVENTION

It is one object of the invention to provide novel and improved compositions comprising an immunoconjugate that comprises an antibody or immunogenic fragment thereof that binds to an antigen expressed by a target cell that is to be eradicated, wherein said antibody or immunogenic fragment thereof possesses human effector function, which antibody or immunogenic fragment thereof is fused at its carboxy terminus to a cytokine that binds a receptor expressed on the surface of a natural killer cell and/or macrophage, thereby resulting in an immunoconjugate that facilitates extracellular (ADCC-type) and intracellular (phagocytic) killing of a target cell, when said immunoconjugate is administered to a host. Preferably, the cytokine is a interferon. Most preferably, the interferon is an α-interferon, especially one which has been FDA approved (e.g., INF-α-2a, INF-α-2b and INF-α-n1).

It is a more specific object of the invention to provide novel immunoconjugates which target cells expressing CD19, CD20, CD22, CD33, CD38, EGF-R, HM 1.24, phosphatidyl serine antigen, HER-2, TAG-72 and MUC1.

It is a further object of the invention to provide novel methods of enhancing apoptosis or treating cancer by administering a therapeutically effective amount of these immunoconjugates to a subject. The target cells to which these immunoconjugates are directed may include malignant cells selected from the group consisting of a breast carcinoma cell, an ovarian carcinoma cell, a prostate carcinoma cell, a lung carcinoma cell, a leukemic T-cell, a leukemic B-cell, a multiple myeloma cell and a B-cell lymphoma cell.

It is another object of the invention to provide a combination therapy to treat a malignancy in a subject comprising an immunoconjugate as described above and at least one chemotherapeutic agent or chemotherapeutic cocktail.

It is a further object of this invention to provide nucleic acids which encode the immunoconjugates described herein.

### DETAILED DESCRIPTION OF THE INVENTION

The present invention relates to IFN-α fusion proteins, specifically immunoconjugates, and the use thereof as therapeutic agents that have the ability to target malignant cells and enhance the killing activity of effector cells through the binding of an IFN-α to its receptor on the effector cell and without systemic administration of IFN-α and its associated toxicity. These immunoconjugates will have enhanced ADCC and phagocytic activity.

I. Definitions

By “interferon-α” or “IFN-α” is meant a protein or fragment thereof which can recognize and bind to the IFN-α receptor. This includes FDA approved forms of IFN-α, such as INF-α-2a (Roferon by Hoffman-LaRoche), INF-α-2b (INTRON® A by Schering Corporation) and INF-α-n1 (lymphoblastoid interferon called Wellferon and produced by Wellcome Foundation Ltd—Wellcome Research Laboratories), as well as other forms of IFN-α (e.g., INF-α-2a and consensus IFN).

The term “fusion protein” as used herein means a hybrid protein produced recombinantly including a synthetic or heterologous amino acid sequence. A fusion protein can be produced from a hybrid gene containing operatively linked heterologous gene sequences.

By “bispecific fusion protein” is meant any immunologically reactive molecule which specifically recognizes and binds two different targets at alternate times or at the same time. In particular, it will refer to an IFN-α antibody fusion protein, wherein the IFN is attached to an antibody, preferably anti-tumor antibody, at the carboxy terminus of the antibody. In a preferred embodiment, the antibody portion may recognize and bind to a target antigen expressed on a targeted cell. In a more preferred embodiment, the antigen-binding, Fc receptor binding, Cl_{q} and C′ activation, and the ability of interferon to bind to its receptor and activate effector cells and macrophages are substantially maintained activities of the immunoconjugate. This is preferably effected by attachment of the interferon directly or indirectly to the antibody hinge, CH1, CH2 or CH3 domain carboxy-terminus.

The terms “effector cell” and “effector function” as used herein means a cell which expresses an IFN-α receptor and can thereby bind to an IFN-α fusion protein. Effector cells can include natural killer (NK) cells, LAK cells, monocytes, macrophages and polymorphonuclear (PMNs) cells. Preferred effector cells include NK cells and macrophages.

An “expression vector” means a nucleic acid molecule comprising (1) a promoter and other sequences (e.g., leader sequences) necessary to direct expression of a desired gene or DNA sequence, and (2) the desired gene or DNA sequence. Optionally, the nucleic acid molecule may comprise a poly A signal sequence to enhance the stability of the gene transcript and/or to increase gene transcription and expression.

By “binding domain,” “binding region” means a binding site which recognizes and binds the entire binding area of a target or any portion thereof. Examples for antibodies or immunoglobulin fragments include: (1) single variable region of an antibody V_{L} or V_{H}; (2) two or more variable regions (e.g., V_{L} and V_{H}, V_{L} and V_{L}; or V_{H} and V_{H}) or the complementary determining region (CDR) thereof; (3) antibody fragments such as Fab₁, Fab₂, SFV, single chain antibodies, domain-deleted antibodies and minibodies; or (4) an IFN-α or a segment of IFN-α which binds to an IFN-α receptor on an effector cell.

By “minibody” is meant an antigen binding protein which includes V_{L} and V_{H} domains of a native antibody fused to the hinge region and CH3 domain of an immunoglobulin or which encodes in a single chain comprising the essential elements of a whole antibody. The single chain comprises the antigen binding region, CH3 domain to permit assembly into a bivalent molecule, and the antibody hinge to accommodate dimerization by disulfide linkages. For methods of preparing minibodies, see, e.g., U.S. Pat. No. 5,837,821.

By “antibody” is intended to refer broadly to any immunologic binding agent such as IgG (including IgG₁, IgG₂, IgG₃, and IgG₄), IgM, IgA, IgD, IgE, as well as antibody fragments. Antibodies in the broadest sense covers intact monoclonal antibodies, polyclonal antibodies, as well as biologically active fragments of such antibodies such as those discussed above. In particular, domain-deleted antibodies are included within the scope of the present invention, such as CH₂ domain-deleted antibodies.

By “monoclonal antibody” is meant an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations, which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature 256: 495 (1975), or may be made by recombinant DNA methods (see, e.g., U.S. Pat. No. 4,816,567). The “monoclonal antibodies” may also be isolated from phage antibody libraries using the techniques described in Clackson et al., Nature 352: 624-628 (1991) and Marks et al., J. Mol. Biol., 222: 581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired therapeutic activity (U.S. Pat. No. 4,816,567; Morrison et al., Proc. Natl. Acad. Sci. USA, 81: 6851-5 (1984)).

“Humanized” forms of non-human (e.g., murine) antibodies are chimeric immunoglobulins, immunoglobulin chains or fragments thereof (such as Fv, Fab, Fab′, F(ab′)₂ or other antigen-binding subsequences of antibodies), which contain minimal sequence derived from a non-human immunoglobulin. For the most part, humanized antibodies are human immunoglobulins (recipient antibody) in which residues from a complementarity-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies may comprise residues which are found neither in the recipient antibody nor in the imported CDR or framework sequences. These modifications are made to further refine and optimize antibody performance. In general the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature, 321: 522-5 (1986); Reichmann et al., Nature, 332: 323-9 (1988); and Presta, Curr. Op. Struct. Biol., 2: 593-6 (1992).

By “target antigen” is meant the antigen recognized by the antibody or immunoglobulin fragment portion of the immunoconjugate. Preferred target antigens include: 5E10, CD1, CD2, CD3, CDCD5, CD7, CD13, CD14, CD15, CD19, CD20, CD21, CD23, CD25, CD33, CD34, CD38, CEA, EGFR, HER-2, HLA-DR, HM 1.24, HMB 45, la, Leu-M1, MUC1, phosphatidyl serine antigen, PMSA and TAG-72.

By “nucleic acid” is meant to include an oligonucleotide, nucleotide, polynucleotide and fragments and portions thereof, and a DNA or a RNA of genomic or synthetic origin, which may be single or double stranded.

By “interferon α” or “IFN-α” preferably is meant to include all members of the interferon-α family of proteins. Fragments of IFN-α are also included, as long as the fragment is capable of recognizing and binding to the IFN-α receptor and thereby activating effector cells. Preferred forms of IFN-α include such FDA approved forms of IFN-α as INF-α-2a, INF-α-2b and INF-α-1n.

By “purified” and “isolated” is meant, when referring to a polypeptide or nucleotide sequence, that the indicated molecule is present in the substantial absence of other biological macromolecules of the same type. The term “purified” as used herein preferably means at least 75% by weight, more preferably at least 85% by weight, more preferably still at least 95% by weight, and most preferably at least 98% by weight, of biological macromolecules of the same type are present. An “isolated nucleic acid molecule which encodes a particular polypeptide” refers to a nucleic acid molecule which is substantially free of other nucleic acid molecules that do not encode the subject polypeptide; however, the molecule may include some additional bases or moieties which do not deleteriously affect the basic characteristics of the composition. Thus, for example, an isolated nucleic acid molecule which encodes a particular CDR polypeptide consists essentially of the nucleotide coding sequence for the subject molecular recognition unit.

By “therapeutically effective” is meant the ability of the immunoconjugate to inhibit in vitro growth of a target cell by greater than about 20%, at a concentration of about 0.1 to about 3.0 μg/ml of the immunoconjugate, wherein said target cells (e.g., tumor cells) are cultured in an appropriate culture medium and said growth inhibition is determined about 4, 5, 6, 7, 8, 9, or 10 days after exposure of the target cells to the immunoconjugate.

By “subject” is meant a living animal or human susceptible to a condition, especially cancer. In the preferred embodiments, the subject is a mammal, including human and non-human mammals. Non-human mammals include dogs, cats, pigs, cows, sheep, goats, horses, rats and mice.

II. Method of Making an IFN-α Fusion Protein

Nucleic acids encoding the desired fusion protein can be inserted into expression vectors for expression. Expression vectors useful in the invention include prokaryotic and eukaryotic expression vectors. Such expression vectors, including plasmids, cosmids, and viral vectors such as bacteriophage, baculovirus, retrovirus and DNA virus vectors, are well known in the art (see, for example, Meth. Enzymol., Vol. 185, D. V. Goeddel, ed. (Academic Press, Inc., 1990), and Kaplift and Loewy (Ed.), Viral Vectors: Gene Therapy and Neuroscience Applications (Academic Press, Inc., 1995), each of which are incorporated herein by reference). Expression vectors contain the elements necessary to achieve constitutive or inducible transcription of a nucleic acid molecule encoding an IFN-α fusion protein. One form of IFN-α is described in Pitha et al., J. Immunol. 141: 3611-6 (1988). Preferred embodiments of the invention will utilize FDA approved forms of IFN-α.

The recombinant nucleic acid molecules encoding an IFN-α fusion protein, an immunoglobulin polypeptide, or a specific IFN-α species or fragment thereof may be obtained by any method known in the art (see, e.g., Maniatis et al., Molecular Cloning: A Laboratory Manual (Cold Spring Harbor Laboratory, Cold Spring Harbor, N.Y., 1982 and 1989) or obtained from publicly available clones, such as pGL2BIFN (ATCC No. 53371) and pALCA1SIFN (ATCC No. 53369). Alternatively, a nucleic acid encoding an IFN-α or an antibody which recognizes a tumor-associated antigen (TAA) may be obtained as follows. A population of cells known to actively express an IFN-α or a specific antibody may be obtained, and total cellular RNA harvested therefrom. The amino acid sequence of the IFN-α or antibody may be used to deduce the sequence of a portion of its nucleic acid so as to design appropriate oligonucleotide primers; or, alternatively, the oligonucleotide primers may be obtained from a known nucleic acid sequence encoding an antibody or an IFN-α. The oligonucleotide fragment may then be used in conjunction with reverse transcriptase to produce a cDNA corresponding to the immunoglobulin and/or IFN-α encoding nucleotide sequence (Okayama et al., Methods Enzymol. 154: 3-29 (1987)). The cDNA can then be cloned, and/or portions of the antibody or IFN-α coding region amplified from this cDNA using polymerase chain reaction (PCR) and appropriate primer sequences (Saiki et al., Science 239: 487-491 (1988)).

In preferred embodiments of the invention, a recombinant vector system may be created to accommodate sequences encoding IFN-α, wherein the IFN-α sequence is attached to the sequences encoding the C-terminus of an antibody or immunogenic fragment that recognizes a tumor-associated antigen. The resultant fusion protein will preserve the ability of the IFN-α molecule to bind to its receptor on the effector cell and enhance the effector cell's killing ability. The immunoconjugate will also preserve the antigen-binding Fc receptor-binding, C1q binding and C′ activation regions of the antibody molecule.

Nucleic acid sequences encoding the various components of the IFN-α based fusion proteins of the invention may be joined together using any techniques known in the art, including restriction enzyme methodologies and the use of synthetic linker sequences.

To provide for adequate transcription of the recombinant constructs of the invention, a suitable promoter/enhancer sequence may be incorporated into the recombinant vector. Promoters which may be used to control the expression of the antibody-based fusion protein include, but are not limited to, the SV40 early promoter region (Bernoist et al., Nature 290: 304-310 (1981)), the promoter contained in the 3′ long terminal repeat of Rous sarcoma viruses (Yamamoto et al., Cell 22: 787-797 (1980)), the herpes thymidine kinase (tk) promoter (Wagner et al., Proc. Natl. Acad. Sci. USA 78: 144-1445 (1981)), the regulatory sequences of the metallothionine gene (Brinster et al., Nature 296: 39-42 (1982)); prokaryotic expression systems such as the LAC, or β-lactamase promoters (Villa-Kamaroff et al., Proc. Natl. Acad. Sci. USA 75: 3727-3731 (1978)), or the tac lambda phage promoter (DeBoer et al., Proc. Natl. Acad. Sci. USA 80: 21-25 (1983)). Other suitable promoters would be apparent to the skilled artisan.

Additionally, it may be desirable to include, as part of the recombinant vector system, nucleic acids corresponding to the 3′ flanking region of an immunoglobulin gene, including RNA cleavage/polyadenylation sites and downstream sequences. Furthermore, it may be desirable to engineer a signal sequence upstream of the IFN-α fusion protein-encoding sequences to facilitate the secretion of the fused molecule from a cell transformed with the recombinant vector.

Creation of IFN-α containing fusion proteins can also utilize sequences encoding conservative amino acid substitutions in an IFN-α sequence, as well as substitutions in the antibody or immunoglobulin region of the fusion protein. Such changes include substituting an isoleucine, valine and leucine for any other of these hydrophobic amino acids; aspartic acid for glutamic acid and vice versa; glutamine for asparagine and vice versa; and serine for threonine and vice versa. Other substitutions can also be considered conservative, depending on the environment of the particular amino acid and its role in the three-dimensional structure of the protein. For example, glycine for alanine can frequently be interchangeable, as well as alanine for valine due to structural and charge similarities.

Successful incorporation of IFN-α based fusion gene constructs may be identified by three general approaches: (a) DNA-DNA hybridization, (b) presence or absence of “marker” gene functions, and (c) expression of inserted sequences. In the first approach, the presence of a foreign gene inserted in an expression vector can be detected by DNA-DNA hybridization using probes comprising sequences that are homologous to the inserted antibody IFN-α fusion protein DNA. In the second approach, the recombinant vector/host system can be identified and selected based upon the presence or absence of certain “marker” gene functions (e.g., thymidine kinase activity, resistance to antibiotics such as G418, transformation phenotype, occlusion body formation in baculovirus, etc.) caused by the insertion of foreign genes in the vector. For example, if the IFN-α fusion gene is inserted so as to interrupt the marker gene sequence of the vector, recombinants containing the antibody fusion gene insert can be identified by the absence of the marker gene function. In the third approach, recombinant expression vectors can be identified by assaying the foreign gene product expressed by the recombinant. Such assays can be based, for example, on the physical or functional properties of the IFN-α fusion gene product in bioassay systems.

The cytokine can be any cytokine or analog or fragment thereof which activates effector cells. The preferred cytokine is an interferon, with the preferred interferon being IFN-α, especially FDA approved forms. The gene encoding the cytokine can be cloned de novo, obtained from an available source, or synthesized by standard DNA synthesis from a known nucleotide sequence as discussed above.

The heavy chain constant region for the conjugates can be selected from any of the five isotypes: alpha (IgA), delta (IgD), epsilon (IgE), gamma (IgG) or mu (IgM). Heavy chains or various subclasses (such as the IgG subclasses 1-4) can be used. The light chains can have either a kappa or lambda constant chain. DNA sequences for these immunoglobulin regions are well known in the art. (See, e.g., Gillies et al., J. Immunol. Meth. 125: 191 (1989)).

In preferred embodiments, the variable region is derived from an antibody specific for the target antigen, and the constant region includes the CH1, CH2 and CH3 domains. The gene encoding the cytokine is joined, in frame to the 3′ end of the gene encoding the constant region (e.g., CH1, CH2 or CH3 exon depending on domain-deleted form desired), either directly or through an intergenic region.

The nucleic acid construct can include an endogenous promoter and enhancer for the variable region-encoding gene to regulate expression of the chimeric immunoglobulin chain. For example, the variable region encoding genes can be obtained as DNA fragments comprising the leader peptide, the VJ gene (functionally rearranged variable (V) regions with joining (J) segment) for the light chain or VDJ gene for heavy chain, and the endogenous promoter and enhancer for these genes. Alternatively, the gene coding for the variable region can be obtained apart from endogenous regulatory elements and used in an expression vector that provides these elements.

Variable region genes can be obtained by standard DNA cloning procedures from cells that produce the desired antibody. Screening of the genomic library for a specific functionally rearranged variable region can be accomplished with the use of appropriate DNA probes, such as DNA segments containing the J region DNA sequence and sequences downstream. Identification and confirmation of correct clones are then achieved by DNA sequencing of the cloned genes and comparison of the sequence to the corresponding sequence of the full length, properly spliced mRNA.

The target antigen preferably can be a cell surface antigen of a tumor cell, but also includes viral antigens or other disease associated antigens expressed on the cell surface. Genes encoding appropriate variable regions can be obtained generally from immunoglobulin producing lymphoid cells. For example, hybridoma cell lines producing immunoglobulin specific for tumor associated antigens or viral antigens can be produced by standard somatic cell hybridization techniques (see, e.g., U.S. Pat. No. 4,96,265.). These immunoglobulin producing cell lines provide the source of variable region genes in functionally rearranged form. The variable region genes will typically be of murine origin, because the murine system lends itself to the production of a wide variety of immunoglobulins of desired specificity.

The DNA fragment containing the functionally rearranged variable region gene is linked to a DNA fragment containing the gene encoding the desired constant region (or a portion thereof). Immunoglobulin constant regions (heavy and light chain) can be obtained from antibody-producing cells by standard gene cloning techniques. Genes for the two classes of human light chains and the five classes of human heavy chains have been cloned, and thus, constant regions of human origin are readily available from publically available clones.

The fused gene encoding the hybrid immunoglobulin heavy chain is assembled or inserted into expression vectors for incorporation into a recipient cell. The introduction of gene construct into plasmid vectors can be accomplished by standard gene splicing procedures.

Recipient cell lines are generally lymphoid cells. The preferred recipient cell is a myeloma (or hybridoma). Myelomas can synthesize, assemble, and secrete immunoglobulins encoded by transfected genes, and they post-translationally modify the protein. A particularly preferred recipient cell is the Sp2/0 myeloma which normally does not produce endogenous immunoglobulin. When transfected, the cell will produce only immunoglobulin encoded by the transfected gene constructs. Transfected myelomas can be grown in culture or in the peritoneum of mice where secreted immunoconjugate can be recovered from ascites fluid. Other lymphoid cells, such as B lymphocytes, also can be used as recipient cells.

There are several methods for transfecting lymphoid cells with vectors containing the nucleic acid constructs encoding the chimeric Ig chain. A preferred way of introducing a vector into lymphoid cells is by spheroblast fusion, as described by Gillies et al., Biotechnol. 7: 798-804 (1989). Alternative methods include electroporation or calcium phosphate precipitation. See also, the methods in Maniatis, Et Al. (1989).

IFN-α based fusion protein produced by the host cell may be collected using any technique known in the art, including, but not limited to, affinity chromatography using target antigen or antibody specific for any portion of the fusion protein. The activity of the fused IFN-α or antibody (e.g., anti-CD20) may be confirmed using biological assays, which detect or measure the activity of the lymphokine or cellular factor. For example, and not by way of limitation, the presence of IFN-α activity may be confirmed in assays which detect receptor binding, virus neutralization and enhanced killing ability of the effector cells.

Preferred methods of detecting such enhanced effector cell ability can utilize receptor binding assays and virus neutralization assays. These assays are described generally below.

Receptor Binding Assay

Receptor binding assays, such as those provided below, can be utilized to determine whether the immunoconjugate binds to the target antigen or to an IFN α receptor.

Virus Neutralization Assay

A virus neutralization assay is one form of receptor binding assay which can be utilized to determine the efficacy of which an immunoconjugate to neutralize virus-infected cells when the antibody targets a viral antigen expressed on the cell surface. For example, virus neutralizations can be determined using the method described by Ho et al., J. Virol. 65: 489-93 (1991), for HIV-1 neutralization using a p24 assay. Neutralization is defined as the percent reduction in the amount of target antigen released into the culture supernatants or detected in cells from wells treated with the immunoconjugate compared with control wells not treated with the immunoconjugate.

ADCC Assay

The ability of the fusion protein to induce ADCC can be assessed using a chromium release assay. Generally, antibodies of the IgG₂ₐ and IgG₃ subclass and occasionally of the IgG₁ subclass mediate ADCC. Antibodies of the IgG₃, IgG₂ₐ and IgM classes bind and activate serum complement. To assess the ability of the immunoconjugates described herein to mediate ADCC and complement activation, one can use a ⁵¹Cr-release assay. Briefly, a cell line expressing the antigen being targeted for lysis by effector cells are labeled with 100 μCi of ⁵¹Cr for about 1 hour prior to combining effector cells and antibodies in a U-bottom microtiter plate. After incubation for about 5 hours at 37° C., supernatants are collected and analyzed for radioactivity. Cytotoxicity can be calculated by the formula: % lysis=[((experimental CPM)−(target leak CPM))/((detergent lysis CPM)−(target leak CPM))]×100%. Specific lysis is calculated using the formula: Specific lysis−(%Lysis with antibody)−(% lysis without antibody).

Complement Binding Assay

To assess the ability of the immunoconjugates to bind complement, the following assay can be utilized. Cells expressing the target antigen recognized by the immunoconjugate are incubated with the immunoconjugate at a concentration of 10 μg/ml. After incubating the plates containing the cells and immunoconjugate for 15 minutes at room temperature, the plates are washed three times. After the third wash, the cells are resuspended in 50 μl of a 1/10 dilution of complement (e.g., guinea pig complement from ICN) and incubated at 37° C. for varying times. Then 50 μl of 0.25% (w/v) trypan blue is added and cell number and plasma integrity of the cells are estimated.

Phagocytosis Assay

To assess the phagocytosis enhancing ability of a particular immunoconjugate, the following assay can be used. Cells expressing the target antigen are labeled with lipophilic red fluorescent dye PKH 26. Buffy coat cells purified from heparinized, whole blood containing effector cells are incubated with the labeled targets at 37° C. for about 6 hours in the absence or in the presence of the immunoconjugate. Effector cells are then stained with FITC (fluorescein isothiocyanate) labeled antibody, which binds to the effector cell at 0° C. Cells are washed and analyzed using two color fluorescence by FACScan or other scanning method. Percent phagocytosis is expressed as the percent of effector cells (NK cells, monocytes, neutrophils or macrophages) that have PKH 26 stain associated with them.

III. Methods of Administering IFN-α Fusion Protein

A fusion protein of the invention is administered to subjects in a biologically compatible form suitable for pharmaceutical administration in vivo. By “biologically compatible form suitable for administration in vivo” is meant a form of the immunogonjugate to be administered in which any toxic effects are outweighed by the therapeutic effects of the protein. An immunogonjugate can be administered in any pharmacological form, optionally in a pharmaceutically acceptable carrier. Administration of a therapeutically effective amount of the immunoconjugate is defined as an amount effective, at dosages and for periods of time necessary to achieve the desired result (e.g., inhibition of the progression or proliferation of the disease being treated). For example, a therapeutically active amount of an immunoconjugate may vary according to such factors as the disease stage (e.g., stage I versus stage IV), age, sex, medical complications, and weight of the individual, and the ability of the immunoconjugate to elicit a desired response in the individual. The dosage regimen may be adjusted to provide the optimum therapeutic response. For example, several divided doses may be administered daily, or the dose may be proportionally reduced as indicated by the exigencies of the therapeutic situation.

The active compound, an immunoconjugate, by itself or in combination with other active agents, such as chemotherapeutic anti-cancer drugs. The immunoconjugate, alone or in combination with other agents, may be administered in a convenient manner such as by injection (subcutaneous, intramuscularly, intravenous, etc.), inhalation, transdermal application or rectal administration. Depending on the route of administration, the active compound may be coated with a material to protect the active compound from the action of enzymes, acids and other natural conditions which may inactivate the compound. A preferred route of administration is by intravenous (I.V.) injection.

To administer an immunoconjugate by other than parenteral administration, it may be necessary to coat the IFN-α fusion protein with, or co-administer the IFN-α fusion protein with, a material to prevent its inactivation. For example, an IFN-α fusion protein can be administered to an individual in an appropriate carrier or diluent, co-administered with enzyme inhibitors or in an appropriate carrier or vector, such as a liposome. Pharmaceutically acceptable diluents include saline and aqueous buffer solutions. Liposomes include water-in-oil-in-water emulsions, as well as conventional liposomes (Strejan et al., J. Neuroimmunol. 7: 27 (1984)). Additional pharmaceutically acceptable carriers and excipients are known in the art or as described in Remington's Pharmaceotical Sciences (18th ed. 1990).

The active compound may also be administered parenterally or intraperitoneally. Dispersions of the active compound can also be prepared in glycerol, liquid polyethylene glycols, and mixtures thereof and in oils. Under ordinary conditions of storage and use, these preparations may contain one or more preservatives to prevent the growth of microorganisms.

Pharmaceutical compositions suitable for injectable use include sterile aqueous solutions (where water soluble) or dispersions and sterile powders for the extemporaneous preparation of sterile injectable solutions or dispersions. In all cases, the composition must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion, and by the use of surfactants. Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols, such as manitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating an active compound (e.g., an IFN-α fusion protein) in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yields a powder of an active ingredient plus any additional desired ingredient from a previously sterile-filtered solution thereof.

When the active compound is suitably protected, as described above, the protein may be orally administered, for example, with an inert diluent or an assimilable edible carrier. As used herein, “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in the therapeutic compositions is contemplated. All compositions discussed above for use with an IFN-α fusion protein may also comprise supplementary active compounds in the composition.

It is especially advantageous to formulate parenteral compositions in dosage unit form for ease of administration and uniformity of a dosage. “Dosage unit form,” as used herein, refers to physically discrete units suited as unitary dosages for the mammalian subjects to be treated; each unit containing a predetermined quantity of active compound is calculated to produce the desired therapeutic effect in association with the required pharmaceutical carrier. The specification for the dosage unit forms of the invention are dictated by and directly dependent on: (A) the unique characteristics of the active compound and the particular therapeutic effect to be achieved; and (B) the limitations inherent in the art of compounding such an active compound for the treatment of sensitivity in individuals.

IV. Method of Treating Cancer

The immunoconjugates described herein can be targeted to a variety of malignant cells which express a tumor-associated antigen (TAA) expressed on the surface of the cell. IFN-α fusion proteins comprising antibodies can be prepared which recognize B and T cell leukemias and lymphomas, multiple myelomas, and solid tumors (e.g., prostate carcinoma, colon carcinoma, lung carcinoma, breast carcinoma and ovarian carcinoma). In preferred embodiments, the immunoglobulin portion of the IFN-α fusion protein may recognize B cell markers (e.g., CD19, CD20, CD22), multiple myeloma antigens (e.g., CD38, HM1.24), leukemia markers (e.g., CD33), and phosphatidyl-serine antigen. Additional markers affiliated with certain malignancies and to which the immunoglobin portion of the fusion protein can include, but are not limited to, the following:

TABLE 1

| Cancer | Tumor Associated Antigens |
|---|---|
| Acute Lymphocytic Leukemia | HLA-Dr, CD34, CD19, CD20, CD1, |
| (ALL) | CD2, CD5, CD7 |
| Acute Myelogenous Leukemia | HLA-Dr, CD34, CD13, CD14, CD15, |
| (AML) | CD33, CD7 |
| Breast Cancer | EGFR, HER-2, MUC1, TAG-72 |
| Carcinoma | CEA, TAG-72, MUC1 |
| Chronic Lymphocytic Leukemia | CD3, CD21, CD20, CD19, CD23, |
| (CLL) | HLA-Dr |
| Hairy Cell Leukemia (HCL) | HLA-Dr, CD19, CD20, CD21, CD25 |
| Hodgkin's Disease | Leu-M1 |
| Melanoma | HMB 45 |
| Non-Hodgkin's Lymphoma | CD20, CD19, Ia |
| Prostate | PSMA, 5E10 |

David A. Scheinberg et al., “The Leukemias” in HARRISON'S PRINCIPLES OF INTERNAL MEDICINE 1764-1774 (Kurt J. lsselbacher, et al., 13^{th} ed. 1994).

V. Drugs to be Used in Combination with the Fusion Protein

The immunoconjugates described above can be used in combination with one or more different cancer treatment modalities, such as radiotherapy, immunotherapy, chemotherapy, and surgery. The combination of treatments used on any particular subject will vary depending on cancer type, stage of disease, family history, age, sex, weight and condition of the subject. Preferably, the immunoconjugates are administered in combination with one more chemotherapeutics. Preferably, chemotherapeutic or chemotherapeutic cocktail is administered in combination with the interferon immunoconjugate described herein, and include those listed in the table below:

TABLE 2

| Cancer | Chemotherapy |
|---|---|
| Acute Lymphocytic | Ara-C alone or with L-asparaginase, doxorubicin, |
| Leukemia (ALL) | idarubicin, mitoxantrone |
| Acute Myelogenous | Hydroxyurea and busulfan; chlorambucil, |
| Leukemia (AML) | melphalan, 6-mercaptopurine, 6-thioguanine, |
| | dibromomannitol, ara-C, IFN-α |
| Breast Cancer | CMF, CAF, CEF, CMFVP, AC, VAT, VATH, |
| | CDDP + VP-16, Tam |
| Chronic Lymphocytic | CAP, CVP, CMP, CHOP |
| Leukemia (CLL) | |
| Hairy Cell Leukemia | 2-chlorodeoxyadenosine, deoxycoformycin, |
| (HCL) | IFN-α |
| Hodgkin's Disease | VABCD, ABDIC, CBVD, PCVP, CEP, EVA, |
| | MOPLACE, MIME, MINE, MTX-CHOP, CEM, |
| | CEVD, CAVP, EVAP, EPOCH, MOPP, MVPP, |
| | ChIVPP, AVD, MOPP + ABVD, MOPP + ABV |
| Melanoma (metastatic) | Dacarbazine, cisplatin, IFN-α-2b, carmustine, |
| | lomustine, tauromustine, fotemustine, carboplatin, |
| | vincristine, vinblastine, vindesine, taxol, |
| | dibromodulcitol, detorubicin, and piritrexim |
| Non-Hodgkin's | IMVP-16, MIME, DHAP, ESHAP, CEPP(B), |
| Lymphoma | CAMP, CHOP, CAP-BOP, CHOP-B, |
| | ProMACE-MOPP, m-BACOD, MACOP-B, |
| | ProMACE-CytaBOM |
| Prostate (hormonally | Doxorubicin, doxorubicin + ketoconazole or |
| relapsed patients) | cyclophosphamide, estramustine, vinblastine, |
| | paclitaxel, navelbine, prenisolone, mitoxantrone |
| | or combinations thereof. |

These chemotherapeutic drugs and drug cocktails (e.g., more than one chemoterapeutic agent) can be administered according to the regimens described in Cancer: Principles & Practice of Oncology (Vincent T. DeVita, Jr. et al., eds., 5^{th} ed. 1997) or as would be known to the skilled artisan.

The abbreviations for the chemotherapeutic cocktail and chemotherapeutic acronyms are as follows:

| | |
|---|---|
| AC | doxorubicin, cyclophosphamide |
| ABDIC | doxorubicin, bleomycin, dacarbazine, lomustine, |
| | prednisone |
| ABVD | doxorubicin, bleomycin, vinblastine, dacarbazine |
| Ara-C | cytarabine |
| AVD | doxorubicin, vinblastine, dacarbazine |
| CAF | cyclophosphamide, doxorubicin, 5-fluorouracil |
| CAMP | lomustine, mitoxantrone, cytarabine, prednisone |
| CAP | cyclophosphamide, doxorubicin, prednisone |
| CAP-BOP | cyclophosphamide, doxorubicin, procarbazine, |
| | bleomycin, vincristine, prednisone |
| CAVP | lomustine, melphalan, etoposide, prednisone |
| CEVD | lomustine, etoposide, vindesine, dexamethasone |
| CDDP + | cisplatin, etoposide, mitomycin C + vinblastine |
| VP-16 | |
| CEF | cyclophosphamide, epirubicin, 5-fluorouracil |
| CEM | lomustine, etoposide, methotrexate |
| CEP | lomustine, etoposide, prednimustine |
| CEPP(B) | cyclophosphamide, etoposide, procarbazine, prednisone, |
| | bleomycin |
| CEVD | lomustine, etoposide, vindesine, dexamethasone |
| ChIVPP | chlorambucil, vinblastine, procarbazine, prednisone |
| CHOP | cyclophosphamide, doxorubicin, vincristine, prednisone |
| CHOP-B | CHOP plus bleomycin |
| CMF | cyclophosphamide, methodtrexate, 5-fluorouracil |
| CMP | cyclophosphamide, melphalan, prednisone |
| CMVP | cyclophosphamide, methotrexate, 5-fluorouracil, |
| | vincristine, prenisone |
| CVP | cyclophosphamide, vincristine, prednisone |
| DHAP | dexamethasone, high-dose Ara-C, cisplatin |
| ESHAP | etoposide, methylpredisolone, high-dose cytarabine, |
| | cisplatin |
| EPOCH | etopsoide, vincristine, doxorubicin, cyclophosphamide, |
| | prednisone |
| EVA | etoposide, vinblastine, doxorubicin |
| EVAP | etoposide, vinblastine, cytarabine, cisplatin |
| IFN-α | interferon α |
| IMVP-16 | ifosfamide, methotrexate, etoposide |
| MACOP-B | methodtrexate, doxorubicin, cyclophosphamide, |
| | vincristine, prednisone, bleomycin, leucovorin |
| m-BACOD | methotrexate, bleomycin, doxorubicin, |
| | cyclophosphamide, vincristine, dexamethasone, |
| | leucovorin |
| MIME | methyl-gag, ifosfamide, methotrexate, etoposide |
| MINE | mitoquazone, ifosfamide, vinorelbine, etoposide |
| MOPLACE | cyclophosphamide, etoposide, prednisone, methotrexate, |
| | cytarabine, vincristine |
| MOPP | nitrogen mustard, vincristine, procarbazine, prednizone |
| MOPP + | MOPP plus doxorubicin, bleomycin, vinblastine |
| ABV | |
| MOPP + | alternating months of MOPP and ABVD |
| ABVD | |
| MVPP | nitrogen mustard, vinblastine, procarbazine, prednisone |
| MTX-CHOP | methotrexate + CHOP |
| PCVP | vinblastine, procarbazine, cyclophosphamide, prednisone |
| ProMACE- | prednisone, doxorubicin, cyclophosphamide, |
| CytaBOM | etoposide, cytarabine, bleomycine, vincristine, |
| | methotrexate, leucovorin |
| ProMACE- | prednisone, methotrexate, doxorubicin, |
| MOPP | cyclophosphamide, etoposide, leucovorin + |
| | standard MOPP |
| Tam | tamoxifen |
| VABCD | vinblastine, doxorubicin, dacarbazine, lomustine, |
| | bleomycin |
| VAT | vinblastine, doxorubicin, thiotepa |
| VATH | vinblastine, doxorubicin, thiotepa, fluoxymesterone |

### EXAMPLES

The examples and methods provided below serve merely to illustrate particular embodiments of the invention and are not meant to limit the invention.

### Example 1

### Immunoconiugate Comprising IFN-α and Rituximab

The nucleic acid encoding an IFN-α (e.g., INF-α-2a, INF-α-2b or INF-α-n1) is operably linked to the nucleic acid encoding Rituximab such that when translated the IFN-α would form the carboxy terminus of the fusion protein. The antigen-binding Fc receptor-binding, C1q binding and complement (C′) activation, as well as the ability of IFN-α to bind to NK cells and macrophages are characteristics possessed by the agents. In addition to the nucleic acid encoding Rituximab, other nucleic acids encoding anti-CD20 antibodies can be operably attached to the nucleic acid encoding IFN-α. The other anti-CD20 antibodies include Ibritumomab, IF5, B1 and 1H4.

### Example 2

### In Vitro Testing of an Immunoconiugate

A tumor cell line (cell expressing a target antigen) expressing Her2/neu (e.g., human breast carcinoma cells, SKBR-3) is selected to determine lysis using the immunoconjugates described herein. Effector cell samples are obtained by using heparinized whole blood or obtained from a third party. To prepare for use as effector cells, monocytes are cultured in Teflon containers in Macrophage Serum-Free Medium (Gibco/BRL) containing 2% human serum for 24 to 48 hours. Target cells are labeled with 100 μCi of ⁵¹Cr for one hour prior to incubation with the effector cells and immunoconjugate in a U-bottomed microtiter plate. After incubation for about 16 to 18 hours at 37° C., supernatants from each well are collected and analyzed for radioactivity. Cytotoxicity and specific lysis can be calculated as previously described.

### Example 3

### In Vivo Testing of Immunoconjugate

For assessment of anti-tumor activity of the immunoconjugates, a mouse model can be used. In the instance of solid tumors, about 1×10⁷ cells in culture media are injected subcutaneously into the right anterior flank of BALB/c, nu/nu or SCID mice. Approximately, fourteen days later or when the tumors have grown to about 0.8 to 1.2 cm in diameter, the mice are separated into groups of 5-10 animals and injected intravenously with 200 μl of immunoconjugate at concentrations of 1 μg to 10 mg. Perpendicular tumor diameters are measured at regular intervals and tumor volumes can be calculated. Alternatively, animals can be euthanized and sections of tumor prepared to determine the impact on tumor progression by the immunoconjugate as compared to the control animals (untreated).

Although the present invention has been described in detail with reference to examples above, it is understood that various modifications can be made without departing from the spirit of the invention. All cited patents and publications referred to in this application are herein incorporated by reference in their entirety.

What is claimed is:

1. An immunoconjugate that comprises an antibody or an immunogenic fragment thereof that binds to an antigen expressed by a target cell that is to be eradicated, wherein said antibody or immunogenic fragment thereof possesses human effector function, which antibody or immunogenic fragment thereof is fused at its carboxy terminus to a cytokine that binds a receptor expressed on the surface of a natural killer cell and/or macrophage, thereby resulting in an immunoconjugate that facilitates extracellular (ADCC-type) and/or intracellular (phagocytic) killing of target cell, when said immunoconjugate is administered to a host.

2. The immunoconjugate of claim 1, wherein the cytokine is an interferon.

3. The immunoconjugate of claim 1, where in the interferon is alpha inteferon (IFN-α).

4. The immunoconjugate of claim 1, where in the interferon is IFN-α and is INF-α-2a, INF-α-2b or INF-α-2n1.

5. The immunoconjugate of claim 1, wherein the target cells are selected from the group consisting of: a breast carcinoma cell, an ovarian carcinoma cell, a prostate carcinoma cell, a lung carcinoma cell, a leukemic T cell, a leukemic B cell, a multiple myeloma cell and a B cell lymphoma cell.

6. The immunoconjugate of claim 1, wherein the antigen is selected from the group consisting of: CD19, CD20, CD22, CD33, CD38, EGF-R, HM1.24, phosphatidyl-serine antigen, HER-2, TAG-72 and MUC-1.

7. The immunoconjugate of claim 6, wherein the antibody which recognizes the antigen CD20 is RITUXAN, IF5, B1 or IH4.

8. The immunoconjugate of claim 6, wherein the antibody which recognizes CD19 is B4, B43 or FVS191.

9. The immunoconjugate of claim 6, wherein the antibody which recognizes CD22 is hLL2, LL2 or RFB4.

10. The immunoconjugate of claim 6, wherein the antibody which recognizes CD33 is M195 or HuM195.

11. The immunoconjugate of claim 6, wherein the antibody which recognizes CD38 is AT13/5.

12. The immunoconjugate of claim 6, wherein the antibody which recognizes HER-2 is HERCEPTIN® or 4D5.

13. The immunoconjugate of claim 6, wherein the antibody which recognizes TAG-72 is HuCC49, HUCC39ΔCH2 or B72.3.

14. The immunoconjugate of claim 6, wherein the antibody which recognizes MUC-1 is 12C10, IG5, H23, BM-2 (2E11), BM-7, 12H12, MAM-6 or HMFG-1.

15. The immunoconjugate of claim 1, wherein the immunogenic fragment is selected from the list consisting of a domain-deleted antibody, Fab, Fab¹, Fab₂, SFV, single chain antibodies, domain-deleted antibodies and minibodies.

16. A method of enhancing apoptosis of a target cell by administering a therapeutically effective amount of an immunoconjugate of claim 1 to a subject.

17. The method of claim 16, wherein the target cell is a malignant cell selected from the group consisting of: a breast carcinoma cell, an ovarian carcinoma cell, a prostate carcinoma cell, a lung carcinoma cell, a leukemic T cell, a leukemic B cell, a multiple myeloma cell and a B cell lymphoma cell.

18. A nucleic acid encoding an immunoconjugate of claim 1.

19. A nucleic acid encoding an immunoconjugate that comprises an antibody or an immunogenic fragment thereof that binds to an antigen expressed by a target cell that is to be eradicated, wherein said an antibody or an immunogenic fragment thereof possesses human effector function, which an antibody or an immunogenic fragment thereof is fused at its carboxy terminus to a cytokine or to a peptide which is fused to a cytokine wherein the that binds a receptor expressed on the surface of a natural killer cell and/or macrophage, thereby resulting in an immunoconjugate that facilitates extracellular (ADCC-type) and intracellular (phagocytic) killing of target cell, when said immunoconjugate is administered to a host.

20. A combination therapy to treat a malignancy in a subject comprising an immunoconjugate of claim 1 and at least one chemotherapeutic agent or chemotherapeutic cocktail.

21. The combination therapy of claim 19, wherein the chemotherapeutic agent is selected from the list consisting of ara-C, doxorubicin, idarubicin, mitoxantrone, chlorambucil, melphalan, 6-mercaptopurine, 6-thioguanine, dibromomannitol, IFN-α, 2-chlorodeoxyadenosine, deoxycoformycin, dacarbazine, cisplatin, carmustine, lomustine, tauromustine, fotemustine, carboplatin, vincristine, vinblastine, vindesine, taxol, dibromodulcitol, detorubicin, piritrexin, estramustine, paclitaxel, navelbine or prenisolone.

22. The combination therapy of claim 19, wherein the chemotherapeutic cocktail is selected from the list consisting of AC, ABDIC, ABVD, Ara-C, AVD, CAF, CAMP, CAP, CAP-BOP, CAVP, CEVD, CDDP+VP-16, CEF, CEM, CEP, CEPP(B), CEVD, ChIVPP, CHOP, CHOP-B, CMF, CMP, CMVP, CVP, DHAP, ESHAP, EPOCH, EVA, EVAP, IFN-α, IMVP-16, MACOP-B, m-BACOD, MIME, MINE, MOPLACE, MOPP, MOPP+ABV, MOPP+ABVD, MVPP, MTX-CHOP, PCVP, ProMACE-CytaBOM, ProMACE-MOPP, VABCD, VAT or VATH.
